(19) United States
(12) Patent Application Publication
(10) Pub. No.: US 2006/0042639 A1
(43) Pub. Date: Mar. 2, 2006

(54) Prophylactic garment

(76) Inventor: Craig Wallace, San Rafael, CA (US)

Correspondence Address: MORRISON & FOERSTER LLP, 425 MARKET STREET, SAN FRANCISCO, CA, 94105-2482, US

(21) Appl. No.: 10/927,744

(22) Filed: Aug. 27, 2004

(51) Int. Cl. A61F 6/06; A61F 6/14

(52) U.S. Cl. 128/830; 128/837; 128/839

(57) ABSTRACT

Described herein is a prophylactic garment for use by a human female wearer. The garment includes an elastomeric portion that provides a barrier against sexually-transmitted diseases, having a substantially form fitting segment configured to cover the perineum, each thigh, and abdomen of the wearer, forming a seal at each thigh opening and the abdomen opening, a tubular segment having an open end integral with the substantially form fitting segment and closed end configured for insertion into the vaginal canal of the wearer, and optionally a tear-resistant perimeter between the form fitting segment and the tubular segment.

### FIELD OF THE INVENTION

The invention relates the field of disease prevention, and more particularly to a prophylactic garment that prevents disease transmission for use by a human female.

### BACKGROUND

According to the United States Center for Disease Control, more than one in five sexually active people in the United States has a sexually transmitted disease. Most of the infected people (more than 65 million) have an incurable disease such as HIV/AIDS, Genital Herpes, Human Papillomavirus (HPV)/genital warts. Since sexually transmitted diseases continue to be spread at the alarming rate of 15 million new infections a year, there needs to be more options available to reduce the rate they are spread. Further, since many of the diseases are incurable, the need does not go away when these people enter committed monogamous relationships—promiscuous sex is not the only factor spreading these diseases.

There are more than 20 known diseases that are spread primarily through sexual activity. Although many of the diseases are spread from fluid transfer (ejaculate, vaginal secretions etc.) the most common diseases (such as genital herpes and human papillomavirus (HPV)) can be spread through contact with ulcerations or skin-to-skin contact. The high-risk, disease spreading, skin contact areas include the genitals, perineum, upper thighs and buttocks.

Standard male and female condoms fall short of providing complete protection of the high-risk areas. Standard male condoms protect the penis and vaginal canal and may stop transfer of fluids between partners if used correctly. However, standard condoms do not provide protection to disease-susceptible areas of the body that are not covered by the condom. Further, many times they are used incorrectly—they require the penis to be withdrawn immediately after ejaculation before softening of the erection occurs, failure to do this often results in exchange of bodily fluids. Similarly, standard female condoms fail to protect the disease-susceptible areas of the body that are not covered by such condoms.

There is a need for a safer, yet attractive and comfortable, barrier type prophylactic. Since the majority of infected people have sexually transmitted diseases that can be passed through skin-to-skin contact, a prophylactic that provides adequate coverage of the high-risk regions would be substantially safer than a design that does not provide such coverage. The device should provide such coverage while still allowing a sufficient level of sensitivity.

### SUMMARY

To satisfy these needs, the present application is directed to a prophylactic garment for use by a human female wearer. The prophylactic garment includes an elastomeric portion that provides a barrier against sexually-transmitted diseases. The elastomeric portion further includes a substantially form fitting segment and a tubular segment. The substantially form fitting segment can be configured to cover the perineum, at least a portion of each thigh, and at least a portion of the abdomen of the wearer, and form a seal at each thigh opening and the abdomen opening. The tubular segment can have an open end integral with the substantially form fitting segment and a closed end configured for insertion into the vaginal canal of the wearer.

In one aspect, the elastomeric portion can be any elastomeric material, such as latex, polyurethane, or silicone elastomer. In one exemplary embodiment, the elastomeric portion has a thickness of greater than 0.005 mm and less than 0.5 mm. The elastomeric portion can be configured to cover the abdomen of the wearer up to at least the crease of the labia majoria of the wearer. The elastomeric portion also can be configured to cover each thigh of the wearer to at least one centimeter below the top of the inside of the thigh of the wearer.

The prophylactic garment can include an absorbent portion placed at each seal at each thigh and abdomen opening.

The present application is also directed to kits including the garment. The kit can include the garment and any other additional component, such as packaging or instructions for use of the garment or an applicator. In a further aspect, the kit can include an agent selected from the group consisting of a spermicide, an antiviral agent, an anti-fungal agent, and anti-bacterial agent, a numbing agent, and a lubrication agent.

### BRIEF DESCRIPTION OF THE DRAWINGS

The present application may be understood by reference to the following description taken in conjunction with the accompanying drawing figures:

FIG. 1A depicts one embodiment of a prophylactic garment according to the invention as worn by a female wearer;

FIGS. 1B-1C depict a second embodiment of a prophylactic garment according to the invention as worn by a female wearer;

FIGS. 1D-1E depict a third embodiment of a prophylactic garment according to the invention as worn by a female wearer;

FIGS. 1F-1H depict a fourth embodiment of a prophylactic garment according to the invention as worn by a female wearer;

FIG. 2A depicts a sectional view of a fifth embodiment of a prophylactic garment according to the invention as worn by a female wearer;

FIG. 2B depicts a sectional view of a sixth embodiment of a prophylactic garment according to the invention as worn by a female wearer;

FIG. 3A depicts a perspective view, with parts broken away, of a tubular section of a prophylactic garment according to yet another embodiment of the invention.

FIG. 3B depicts a perspective view, with parts broken away, of a tubular section of a prophylactic garment according to yet another embodiment of the invention.

FIG. 3C depicts a perspective view, with parts broken away, of a tubular section of a prophylactic garment according to yet another embodiment of the invention

FIG. 4 depicts a perspective view, of a tear resistant perimeter of a prophylactic garment according to yet another embodiment of the invention

### DETAILED DESCRIPTION

The present invention is directed to a prophylactic garment for use by a human female wearer. The prophylactic garment serves as a barrier type contraceptive and disease prevention garment that may be worn by a female prior to and/or during sexual activity. The garment may be worn for numerous hours prior to sexual activity. The garment provides coverage of the genital areas of the female as well as coverage of potentially disease spreading areas of the upper thighs and buttocks. In particular, the garment limits skin-to-skin contact in the high-risk genital and surrounding areas of the body, as well as fluid transfer between sexual partners. The garment offers a high level of protection while still remaining visibly appealing, comfortable and providing sufficient sensitivity to the users. The garment may be used, for example, in sexual acts including but not limited to oral sex and sexual intercourse. The garment may be made in different sizes, or one size fits all, depending on material used.

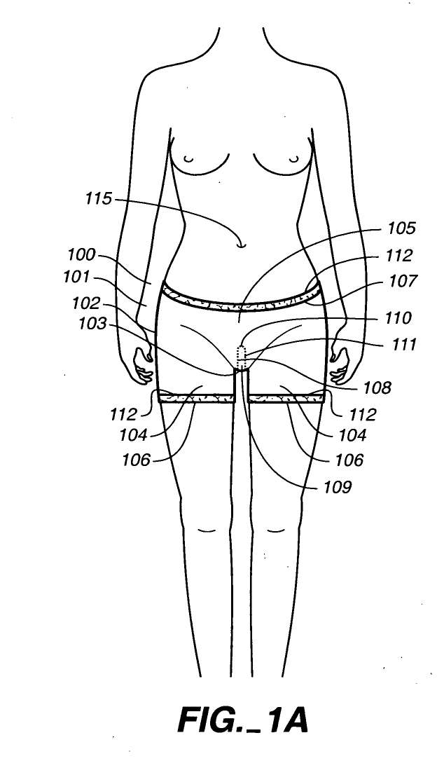

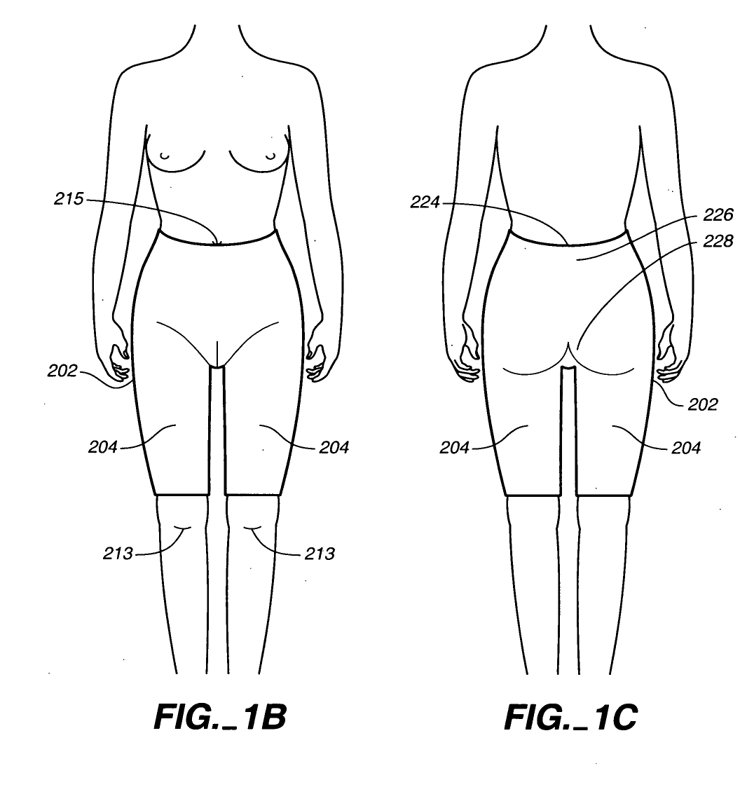

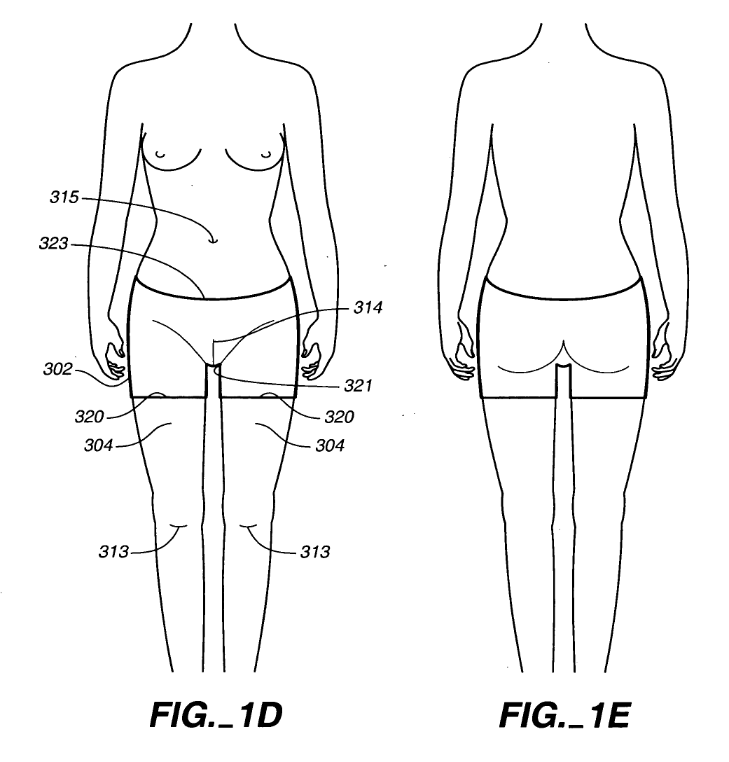

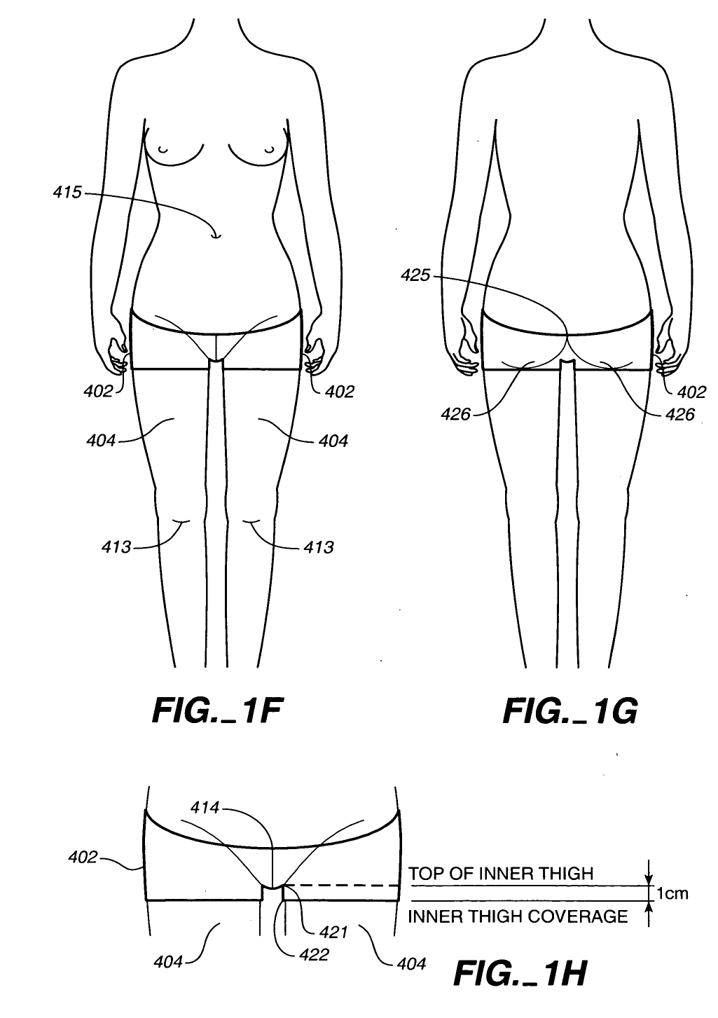

With reference to an embodiment of the invention depicted in FIG. 1A, prophylactic garment 100 includes elastomeric portion 101 having a substantially form fitting segment 102 configured to cover the perineum 103, each thigh 104, and abdomen 105 of the wearer. Substantially form fitting segment 102 is further configured to form a thigh seal 106 at each thigh opening and a abdomen seal 107 at the abdomen opening of the garment. Prophylactic garment 100 further includes tubular segment 108 having open end 109 and closed end 110. Open end 109 of tubular segment 108 is integral with substantially form fitting segment 102, and closed end 110 is configured for insertion into the vaginal canal 111 of the wearer. Prophylactic garment 100 also includes an absorbent portion 112 placed at each thigh seal 106 and abdomen seal 107.

The elastomeric portion of the garment includes a substantially form fitting segment specifically designed for a woman's form. The substantially form fitting segment can be designed to be in the general shape of a woman's brief, boxer shorts, bicycle shorts or Bermuda shorts. The substantially form fitting segment is thus configured to cover at least a portion of the thigh region, at least a portion of the buttock region, and at least a portion of the abdominal region of the wearer. Open ulcerations or lesions that may be hidden by the labia majora or nearby pubic hair are not exposed to contact with a sexual partner when the garment is worn.

The substantially form fitting segment is configured to cover the perineum and at least a portion of each thigh of the wearer. Specifically, the substantially form fitting segment may be configured to cover any region of the thigh of a wearer. With reference to the embodiment depicted in FIGS. 1B-1C, the substantially form fitting segment 202 is configured to cover the thigh region 204 of the wearer to the top of the kneecaps 213. With reference to the embodiment depicted in FIGS. 1D-1E, the substantially form fitting segment 302 is configured to cover the thigh region 304 of the wearer to a point 320 between the top of the inside of the thigh 321 and the top of the kneecaps 313. With reference to the embodiment depicted in FIGS. 1F-1H, the substantially form fitting segment 402 is configured to cover the thigh region 404 of the wearer to a point 422 at least one centimeter below the top of the inside of the thigh 421.

The substantially form fitting segment is also configured to cover at least a portion of the abdomen of the wearer. With reference to FIGS. 1F-1H, in one embodiment of the invention, the front portion of substantially form fitting segment 402 may be configured to provide frontal coverage at least equal to the top (in standing position) of the crease formed by the left and right labia majora 414 of the wearer. With reference to FIGS. 1D-1E, in another embodiment of the invention, the front portion of the substantially form fitting segment 302 may be configured to extend to some point 323 between the top (in standing position) of the crease formed by the left and right labia majora 314 of the wearer and the belly button 315 of the wearer. With reference to FIGS. 1B-1C, in another embodiment of the invention, the front portion of the substantially form fitting segment 202 may be configured to extend to the belly button 215 of the wearer.

With reference to FIGS. 1F-1H, in one embodiment of the invention, the rear portion of the substantially form fitting segment 402 may be configured to cover a region of the buttocks to a point 425 at least equal to the top of the vertical crease formed by the left and right gluteus maximus muscles 426. The substantially form fitting segment 402 may be configured to cover the entire left and right gluteus maximus muscles 426. The rear portion of the substantially form fitting segment may be configured to extend up the back of the wearer. With reference to FIGS. 1B-1C, in one embodiment of the invention, the substantially form fitting segment 202 is configured to extend up the back 226 of the wearer to a point 224 equal to the horizontal plane of the belly button 215. This embodiment provides additional coverage of the potentially disease spreading buttocks area 228. The rear portion of the substantially form fitting segment 202 may be configured to extend down the thighs 204 of the wearer.

The substantially form fitting segment can be further configured to include a seal at one or more of each thigh opening and/or the abdomen opening. The seal(s) may be configured to reduce ingress and egress of fluids in the garment, prevents breakage of the garment, and/or prevents the garment from slipping. Each seal may be of any material known in the art. In one embodiment, the seal is configured to be formed directly from the same material as the substantially form fitting segment. In another embodiment, each seal may include an additional material to the elastomeric material, including but not limited to elastic, string, Velcro, or one or more buckles. The additional security material may be configured at one or more thigh opening and/or abdominal opening of the garment. An agent, such as a spermicidal agent, antiviral agent, antibacterial agent, or antifungal agent, may be added at one or more of the seals.

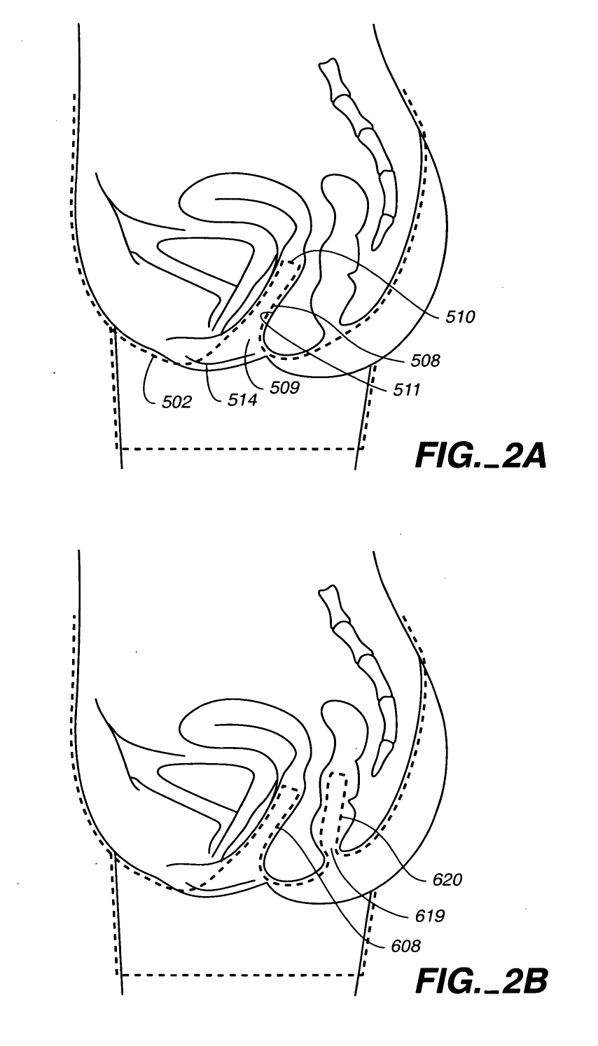

With reference to the embodiment depicted in FIG. 2A, the elastomeric portion of the garment also includes tubular segment 508 having open end 509 integral with the substantially form fitting segment 502 and closed end 510 configured for insertion into the vaginal canal 511 of the wearer. Tubular segment 508 is integral to and non-detachable from the substantially form fitting segment 502, and is adapted for insertion into the vaginal canal 511 prior to sexual activity. This tubular segment 508 is configured to line the wearer's vaginal canal 511.

The location and angle of the tubular segment is specifically configured for use by a human female wearer, not a male human wearer. In particular, the tubular segment is positioned on the garment to correspond to the entrance to a female wearer's vaginal canal when the garment is worn. The tubular segment is further configured to correspond to the angle of a woman's vaginal canal. This contrasts with a garment designed for the male anatomy, in which the male penis is located in a position anterior to the location that corresponds to the female vaginal canal.

The tubular segment may be of any shape, including, but not limited to, a cylinder or a cone. The tubular segment is longer (measured from open end to closed end) than it is wide (measured from each side of tubular segment). The opening of the open end of the tubular segment may be any shape, including, but not limited to, an oval, a circle, a slit, or any other shape. Preferably, the inner diameter of the tubular segment is configured to be large enough to allow the movement of an erect penis during sexual intercourse.

A finger, an applicator type device, or any other suitable method may be used to insert the tubular segment into the vaginal canal. The interior or exterior of the tubular segment may optionally be lubricated before or after insertion. Prior to intercourse the inner portion of the tubular segment is preferably lubricated to allow an erect penis to move within it without substantial binding.

In another aspect of the invention, one or more flexible rings may be used to secure the tubular segment in the vaginal canal of the wearer. The diameter dimension of each flexible ring may be larger than the diameter of the tubular segment. One or more flexible rings may be configured to be positioned just past the entrance to the vaginal canal where the vaginal canal is typically larger than the entrance. Alternatively, the flexible ring may be located near the closed end of the tubular segment. In another embodiment, the one or more integral rings may be used at any point along the vaginal canal.

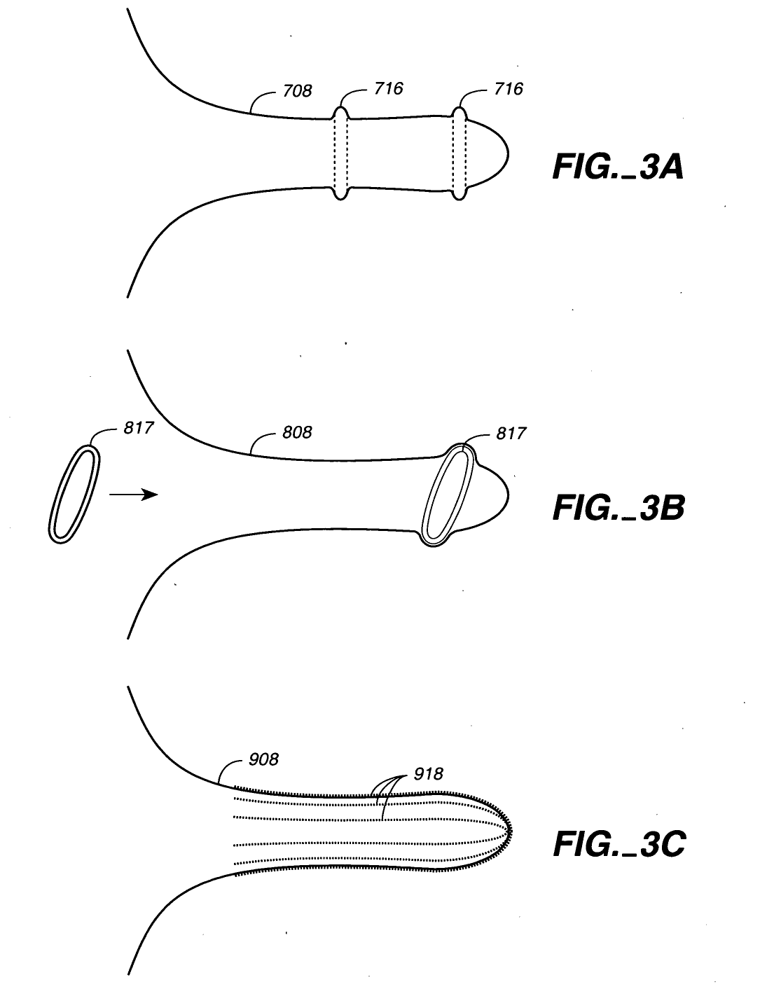

FIG. 3A depicts a tubular segment with integral rings. One or more rings 716 may be integral and part of tubular segment 708. If rings 708 are integral to the tubular segment, they are constructed as a part of the tubular segment. FIG. 3B depicts a tubular segment with non-integral rings. One or more rings 817 are non-integral and separate from tubular segment 808. Non-integral rings 817 may be positioned at any location along the tubular segment 808 chosen by the wearer. The rings may also be used to create additional surface tension in any part of the vaginal canal to help limit movement of the tubular segment in the vagina.

Such rings may be constructed of any suitable natural or synthetic material such as latex, polyurethane, wire, spring or silicone elastomer or combination. Alternatively, the flexible ring may be made of wire or spring type material, and optionally coated with a flexible material. Different flexible rings may be constructed of the same or different material.

In another aspect of the invention, the side of the tubular segment that contacts the vaginal canal may be textured according to methods known in the art to further aid in securing the tubular segment in the vaginal canal of the wearer. The texture would create additional surface tension in the vaginal canal to help limit movement of the tubular segment in the vagina. FIG. 3C depicts such an embodiment. Texture 918 is put at any point along the tubular segment 908.

In a further aspect, an agent may be placed on one or more sides of the tubular segment. The agent may be, but is not limited to, a spermicidal agent, an antiviral agent, an antibacterial agent, an anti-fungal agent, a lubricating agent, or a numbing agent. Any spermicidal agent, antiviral agent, antibacterial agent, anti-fungal agent, lubricating agent, or numbing agent may be used. The agent may be placed on the tubular segment prior to or after the wearer puts on the garment. Alternatively, the agent may be placed on the tubular segment immediately prior to sexual activity.

The elastomeric portion of the garment may be constructed of a material that is elastic enough to sufficiently compensate for any misalignment of the tubular segment due to slight operator error or variances in body type. The material of the substantially form fitting segment may be flexible and elastic enough to allow the garment to be substantially form fitting. In one aspect, the elastomeric portion provides a barrier against sexually-transmitted diseases. The elastomeric portion may be constructed of any flexible, elastic, natural or synthetic material known in the art, including but not limited to latex, polyurethane, or silicone elastomer. The elastomeric portion may also be hypoallergenic. The material serves as a barrier to transmission of particles of similar size to those of the smallest sexually transmitted disease viruses. The elastomeric portion is constructed from a material having a reasonable shelf life without decay. The material may be smooth enough to allow a sufficient level of comfort and strong enough to sufficiently avoid breakage.

In a further embodiment of the invention, the entire elastomeric portion of the garment is made without seams as a single piece of latex. Such a design can be made by dipping a former in the general shape of a woman's lower body into liquid latex in a manner similar to that used to make latex gloves. This process of making the garment is economic enough to compete with existing barrier type prophylactics and generally considered far more economical to any process that requires stitching or gluing. Since it is very unlikely that the regulatory agencies will approve a reuseable disease prevention barrier type prophylactic, the cost of each unit is critical for widespread acceptance.

In another embodiment of the invention, the elastomeric portion is form fitting and shaped to the extent that it parts, or separates, the left and right labia majora and/or left and right gluteus maximus muscles. For example, the garment can be designed with a pronounced “V” shape in the crotch and/or buttocks region to achieve such a result. Such a design could enhance the appearance of the garment by more closely representing the appearance of wearing no clothes. Further, such a design would allow improved access to sensitive areas such as the clitoris or anus of the wearer.

In another aspect of the invention, the material of the elastomeric portion may be less than 0.5 mm thick to provide adequate sensitivity to the user and her partner. Additionally, the material may be more than 5 microns (0.005 mm) to provide adequate strength and integrity of the design. Different areas of the elastomeric portion may have different thicknesses. For example, the substantially form fitting segment may have a different thickness than the tubular segment. Alternatively, different areas of the substantially form fitting segment may have different thicknesses.

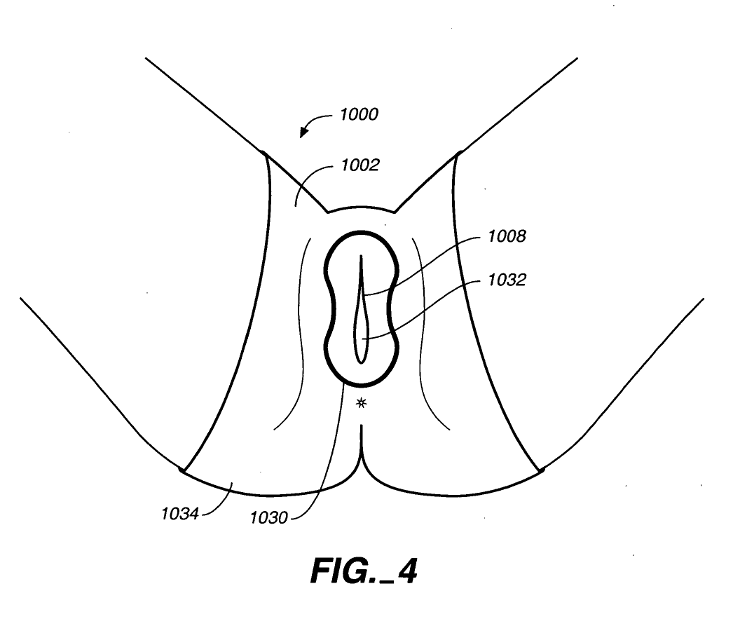

In another embodiment of the invention there is a reinforced tear resistant perimeter located between the substantially form fitting segment and the tubular segment. FIG. 4 depicts a tear resistant perimeter 1030 surrounding the vagina 1032 and tubular segment 1008. The tear resistant perimeter 1030 prevents damage that may occur in the substantially form fitting segment 1002 of the garment 1000 from continuing into the tubular section 1008 of the garment. For example, if a tear was to occur in the buttocks area 1034 of the substantially form fitting segment 1002, the tear would not likely continue beyond the tear resistant perimeter 1030 into the tubular section 1008. Although the protection offered by the garment 1000 would be partially compromised, it would still offer a significant level of protection.

The location and design of the tear resistant perimeter 1030 can reasonably prevent it from entering the vaginal canal or anal cavity of the wearer. This helps ensure that the vaginal canal or anal cavity remains properly lined even if the surrounding substantially form fitting segment 1002 is damaged.

The tear resistant perimeter can also limit stretching of the area within the tear resistant perimeter. The portion within the reinforced perimeter would not be substantially stretched due to fitment of the rest of the garment. This would help ensure a standardized thickness and therefore level of protection of that critical part of the garment.

The reinforced perimeter can be formed of a variety of materials. In one embodiment the reinforced perimeter is made of an inelastic natural or synthetic fiber such as cotton string or Kevlar. In another embodiment the reinforced perimeter is made of an elastic material such as latex or silicon elastomer. In another embodiment the reinforced perimeter is made of polyurethane.

In another aspect of the invention, the garment may include multiple layers of material for added thickness. The added layers may be at the substantially form fitting segment, the tubular segment, or both the substantially form fitting segment and tubular segment. The added thickness results in improved strength and/or function of the garment. In one aspect of the invention, the thickness is less than 2 mm total.

The garment may also include an absorbent portion. In one aspect of the invention as depicted in FIG. 1A, absorbent portion 112 is placed at one or more seals (106, 107) of the substantially form fitting segment 102 of the garment. The garment may be configured to align absorbent portion 112 on the exterior of a seal (106, 107) of substantially form fitting portion 102. Alternatively, the absorbent portion may be configured to be on the interior of the substantially form fitting portion.

The absorbent portion can absorb fluid present at a seal. The absorbent portion thus further reduces the ingress or egress of fluid, and exposure of the skin of the wearer to any sexually transmitted disease particles. In one configuration, the absorbent portion absorbs ejaculate prior to removal of the garment to avoid spillage.

The absorbent portion may be constructed of any absorbent material known in the art. Some portion of the substantially form fitting segment may include an absorbent material to absorb moisture produced while wearing the garment.

In further aspects of the invention, the garment may be configured to have a second tubular segment positioned for penetrating the anal cavity of the wearer. FIG. 2B depicts a sectional view with a second tubular segment 620. The second tubular segment 620 is shown inside the anus 619 of the wearer. The second tubular segment 620 may have one or more of the characteristics of the first tubular segment 608, as discussed in detail above.

The second tubular segment may be of any shape, including, but not limited to, a cylinder or cone. One or more integral or non-integral flexible rings may be provided with the second tubular segment. The second tubular segment may also be textured. The second tubular segment may be made out of any barrier against sexually-transmitted diseases known in the art. The second tubular segment may be constructed of a material that is the same as or different than the substantially form fitting region or the first tubular segment.

The prophylactic garment may include a coating for ease of application. The coating may be a powder coating. Any powder, such as an inert talc based powder, may be used. Alternatively, any liquid or gel compound, such as aloe, may be used.

The garment may be lubricated. The tubular segment may be lubricated prior the wearer putting on the garment. Alternatively, the tubular segment or may be lubricated after the wearer has put on the garment. Any lubricant known in the art may be used.

The garment may further include one or more pockets. These pockets may be located at any position on the garment, such as the anterior of the thigh region, buttock, region, abdomen, or front of the thigh. These pockets may be configured to hold any device or agent, such as a prophylactic, sexual aid, spermicide agent, antiviral agent, anti-fungal agent, anti-bacterial agent, numbing agent, or lubricant. Such a pocket may be configured to contain the garment when folded, rolled, or stuffed into the pocket. Pockets may be configured to be sealed closed by any sealing method known in the art.

The substantially form fitting portion of the garment may further include one or more openings for ventilation. The openings may be positioned away from the location of the first tubular segment and second tubular segment.

The garment may also include decorative elements. The garment may be any color, including transparent, translucent, black, white or any other color or shade. The garment may have a pattern or design. The garment may also include one or more loosely fitting sections for improved looks. The garment may be flavored with a natural and/or artificial flavor, or scented with a natural and/or artificial scent.

The garment may be configured to be rolled on or pulled on prior to use. The garment may be configured to be discarded after use, or cleaned and reused. In one aspect, the garment may be provided with a cleaning solution allowing the garment to be cleaned and reused.

In another aspect, the prophylactic garment may be packaged as part of a kit. The kit may include any embodiment of the garment, as described in detail above. The kit may further include an agent such as a spermicide, an antiviral agent, an anti-fungal agent, and anti-bacterial agent, a numbing agent, or a lubrication agent. The kit may also include instructions for its use. Optionally, the agent may be contained in a package separate from the garment. The kit may include any aspect, embodiment, or variation of the garment, as described above. The kit may also include an applicator device designed to insert the first tubular segment into the vaginal canal or second tubular segment into the anus of the wearer.

Although the present invention has been described with respect to certain embodiments, configurations, examples, and applications, it will be apparent to those skilled in the art that various modifications and changes may be made without departing from the invention.

I claim:

1. A prophylactic garment for use by a human female wearer comprising:
an elastomeric portion that provides a barrier against sexually-transmitted diseases, said elastomeric portion including
a substantially form fitting segment configured to cover the perineum, at least a portion of each thigh, and at least a portion of the abdomen of the wearer; and
a tubular segment having an open end integral with the substantially form fitting segment and a closed end configured for insertion into the vaginal canal of the wearer;
and wherein said elastomeric portion has a thickness of greater than 0.005 mm and less than 0.5 mm.

2. The garment of claim 1, wherein the elastomeric portion is configured to cover at least a portion of each thigh of the wearer down to at least one centimeter below the top of the inside of the thigh.

3. The garment of claim 1, wherein the elastomeric portion is configured to cover the abdomen of the wearer up to at least the crease of the labia majora of the wearer.

4. The garment of claim 1, wherein the elastomeric portion is configured to cover a region of the buttocks up to a point at least equal to the top of the vertical crease formed by the left and right gluteus maximus muscles.

5. The garment of claim 1, further comprising a seal at each thigh opening and the abdomen opening.

6. The garment of claim 5, wherein each said seal comprises an elastic band.

7. The garment of claim 1, further comprising one or more flexible retention rings having a diameter equal to or greater than the diameter of the tubular segment, wherein the one or more flexible retention rings are configured to secure the tubular segment in the vagina of the wearer.

8. The garment of claim 1, wherein the tubular segment has raised texture configured to secure the tubular segment in the vagina of the wearer.

9. The garment of claim 1, further comprising an agent selected from the group consisting of a spermicidal agent, an antiviral agent, an anti-fungal agent, an anti-bacterial agent, a lubricating agent and a numbing agent.

10. The garment of claim 1, wherein the elastomeric portion is latex, polyurethane, or silicone elastomer.

11. The garment of claim 1 is made entirely without seams from a single continuous piece of latex.

12. The garment of claim 1, wherein the elastomeric portion is form fitting to the extent that it parts the left and right labia majora and left and right gluteus maximus muscles of the wearer.

13. The garment of claim 1, wherein the elastomeric portion includes a tear resistant perimeter located between the substantially form fitting segment and the tubular segment.

14. The garment of claim 13, wherein the tear resistant perimeter is designed to resist entry into the vaginal canal or anal cavity of the wearer.

15. The garment of claim 13, wherein the tear resistant perimeter is designed to limit stretching of the area within the perimeter caused by forces outside the perimeter.

16. The garment of claim 5, further comprising an absorbent portion placed at each seal at each thigh and abdomen opening.

17. The garment of claim 1, further comprising an additional tubular section aligned at the entrance to the anal cavity of the wearer.

18. The garment of claim 1, further comprising one or more pockets.

19. The garment of claim 1, further comprising one or more ventilation openings.

20. A kit comprising:
the garment of claim 1, and one or more of the following items:
an agent selected from the group consisting of a spermicide, an antiviral agent, an anti-fungal agent, and anti-bacterial agent, a numbing agent, and a lubrication agent, and
instructions for proper use, and
a vaginal applicator, and
an anal applicator.

21. A prophylactic garment for use by a human female wearer comprising:
an elastomeric portion that provides a barrier against sexually-transmitted diseases, said elastomeric portion including
a substantially form fitting segment configured to cover the perineum, at least a portion of each thigh, and at least a portion of the abdomen of the wearer; and
a tubular segment having an open end integral with the substantially form fitting segment and a closed end configured for insertion into the vaginal canal of the wearer.
a tear resistant perimeter located between the substantially form fitting segment and the tubular segment.

22. The garment of claim 21, wherein the elastomeric portion is configured to cover at least a portion of each thigh of the wearer down to at least one centimeter below the top of the inside of the thigh.

23. The garment of claim 21, wherein the elastomeric portion is configured to cover the abdomen of the wearer up to at least the crease of the labia majora of the wearer.

24. The garment of claim 21, wherein the elastomeric portion is configured to cover a region of the buttocks up to a point at least equal to the top of the vertical crease formed by the left and right gluteus maximus muscles.

25. The garment of claim 21, further comprising a seal at each thigh opening and the abdomen opening.

26. The garment of claim 25, wherein each said seal comprises an elastic band.

27. The garment of claim 21, further comprising one or more flexible retention rings having a diameter equal to or greater than the diameter of the tubular segment, wherein the one or more flexible retention rings are configured to secure the tubular segment in the vagina of the wearer.

28. The garment of claim 21, wherein the tubular segment has raised texture configured to secure the tubular segment in the vagina of the wearer.

29. The garment of claim 21, further comprising an agent selected from the group consisting of a spermicidal agent, an antiviral agent, an anti-fungal agent, an anti-bacterial agent, a lubricating agent and a numbing agent.

30. The garment of claim 21, wherein the elastomeric portion is latex, polyurethane, or silicone elastomer.

31. The garment of claim 21 is made entirely without seams from a single continuous piece of latex.

32. The garment of claim 21, wherein the elastomeric portion is form fitting to the extent that it parts the left and right labia majora and left and right gluteus maximus muscles of the wearer.

33. The garment of claim 21, wherein the elastomeric portion has a thickness of greater than 0.005 mm and less than 0.5 mm.

34. The garment of claim 21, wherein the tear resistant perimeter is designed to resist entry into the vaginal canal or anal cavity of the wearer.

35. The garment of claim 21, wherein the tear resistant perimeter is designed to limit stretching of the area within the perimeter caused by forces outside the perimeter.

36. The garment of claim 25, further comprising an absorbent portion placed at each seal at each thigh and abdomen opening.

37. The garment of claim 21, further comprising an additional tubular section aligned at the entrance to the anal cavity of the wearer.

38. The garment of claim 21, further comprising one or more pockets.

39. The garment of claim 21, further comprising one or more ventilation openings.

40. A kit comprising:
the garment of claim 21, and one or more of the following items:
an agent selected from the group consisting of a spermicide, an antiviral agent, an anti-fungal agent, and anti-bacterial agent, a numbing agent, and a lubrication agent, and
instructions for proper use, and
a vaginal applicator, and
an anal applicator.

41. A prophylactic garment for use by a human female wearer comprising:
an elastomeric portion that provides a barrier against sexually-transmitted diseases, said elastomeric portion including
a substantially form fitting segment configured to cover the perineum, at least a portion of each thigh, and at least a portion of the abdomen of the wearer, and to form a seal at each thigh opening and the abdomen opening, and further having an absorbent portion placed at each seal at each thigh and abdomen opening; and
a tubular segment having an open end integral with the substantially form fitting segment and a closed end configured for insertion into the vaginal canal of the wearer.

42. The garment of claim 41, wherein the elastomeric portion is configured to cover at least a portion of each thigh of the wearer down to at least one centimeter below the top of the inside of the thigh.

43. The garment of claim 41, wherein the elastomeric portion is configured to cover the abdomen of the wearer up to at least the crease of the labia majora of the wearer.

44. The garment of claim 41, wherein the elastomeric portion is configured to cover a region of the buttocks up to a point at least equal to the top of the vertical crease formed by the left and right gluteus maximus muscles.

45. The garment of claim 41, wherein each said seal comprises an elastic band.

46. The garment of claim 41, further comprising one or more flexible retention rings having a diameter equal to or greater than the diameter of the tubular segment, wherein the one or more flexible retention rings are configured to secure the tubular segment in the vagina of the wearer.

47. The garment of claim 41, wherein the tubular segment has raised texture configured to secure the tubular segment in the vagina of the wearer.

48. The garment of claim 41, further comprising an agent selected from the group consisting of a spermicidal agent, an antiviral agent, an anti-fungal agent, an anti-bacterial agent, a lubricating agent and a numbing agent.

49. The garment of claim 41, wherein the elastomeric portion is latex, polyurethane, or silicone elastomer.

50. The garment of claim 41, wherein the elastomeric portion is form fitting to the extent that it parts the left and right labia majora and left and right gluteus maximus muscles of the wearer.

51. The garment of claim 41, wherein the elastomeric portion has a thickness of greater than 0.005 mm and less than 0.5 mm.

52. The garment of claim 41, wherein the elastomeric portion includes a tear resistant perimeter located between the substantially form fitting segment and the tubular segment.

53. The garment of claim 52, wherein the tear resistant perimeter is designed to resist entry into the vaginal canal or anal cavity of the wearer.

54. The garment of claim 52, wherein the tear resistant perimeter is designed to limit stretching of the area within the perimeter caused by forces outside the perimeter.

55. The garment of claim 41, further comprising an additional tubular section aligned at the entrance to the anal cavity of the wearer.

56. The garment of claim 41, further comprising one or more pockets.

57. The garment of claim 41, further comprising one or more ventilation openings.

58. A kit comprising:
the garment of claim 41, and one or more of the following items:
an agent selected from the group consisting of a spermicide, an antiviral agent, an anti-fungal agent, and anti-bacterial agent, a numbing agent, and a lubrication agent, and
instructions for proper use, and
a vaginal applicator, and
an anal applicator

59. A prophylactic garment for use by a human female wearer comprising:
an elastomeric portion that provides a barrier against sexually-transmitted diseases, said elastomeric portion including
a substantially form fitting segment configured to cover the perineum, at least a portion of each thigh, and at least a portion of the abdomen of the wearer; and
a tubular segment having an open end integral with the substantially form fitting segment and a closed end configured for insertion into the vaginal canal of the wearer; and
wherein the elastomeric portion is form fitting to the extent that it parts the left and right labia majora and left and right gluteus maximus muscles of the wearer.

60. The garment of claim 59, wherein the elastomeric portion is configured to cover at least a portion of each thigh of the wearer down to at least one centimeter below the top of the inside of the thigh, and down to at most the top of the kneecaps of the wearer.

61. The garment of claim 59, wherein the elastomeric portion is configured to cover the abdomen of the wearer up to at least the crease of the labia majora of the wearer.

62. The garment of claim 59, wherein the elastomeric portion is configured to cover a region of the buttocks up to a point at least equal to the top of the vertical crease formed by the left and right gluteus maximus muscles.

63. The garment of claim 59, further comprising a seal at each thigh opening and the abdomen opening.

64. The garment of claim 63, wherein each said seal comprises an elastic band.

65. The garment of claim 59, further comprising one or more flexible retention rings having a diameter equal to or greater than the diameter of the tubular segment, wherein the one or more flexible retention rings are configured to secure the tubular segment in the vagina of the wearer.

66. The garment of claim 59, wherein the tubular segment has raised texture configured to secure the tubular segment in the vagina of the wearer.

67. The garment of claim 59, further comprising an agent selected from the group consisting of a spermicidal agent, an antiviral agent, an anti-fungal agent, an anti-bacterial agent, a lubricating agent and a numbing agent.

68. The garment of claim 59, wherein the elastomeric portion is latex, polyurethane, or silicone elastomer.

69. The garment of claim 59 is made entirely without seams from a single continuous piece of latex.

70. The garment of claim 59, wherein the elastomeric portion has a thickness of greater than 0.005 mm and less than 0.5 mm.

71. The garment of claim 59, wherein the elastomeric portion includes a tear resistant perimeter located between the substantially form fitting segment and the tubular segment.

72. The garment of claim 71, wherein the tear resistant perimeter is designed to resist entry into the vaginal canal or anal cavity of the wearer.

73. The garment of claim 71, wherein the tear resistant perimeter is designed to limit stretching of the area within the perimeter caused by forces outside the perimeter.

74. The garment of claim 63, further comprising an absorbent portion placed at each seal at each thigh and abdomen opening.

75. The garment of claim 59, further comprising an additional tubular section aligned at the entrance to the anal cavity of the wearer.

76. The garment of claim 59, further comprising one or more pockets.

77. The garment of claim 59, further comprising one or more ventilation openings.

78. A kit comprising:
the garment of claim 59, and one or more of the following items:
an agent selected from the group consisting of a spermicide, an antiviral agent, an anti-fungal agent, and anti-bacterial agent, a numbing agent, and a lubrication agent, and
instructions for proper use, and
a vaginal applicator, and
an anal applicator.
